(19) United States
(12) Patent Application Publication
(10) Pub. No.: US 2019/0274546 A1
(43) Pub. Date: Sep. 12, 2019

(54) METHOD AND APPARATUS FOR DETERMINING PROPERTIES OF AN EYE

(71) Applicant: The University of Liverpool, Liverpool, Merseyside (GB)

(72) Inventors: Ahmed ELSHEIKH, Liverpool (GB); Brendan John GERAGHTY, Liverpool (GB); John Haldyn CLAMP, Cambridge (GB)

(21) Appl. No.: 16/463,793

(22) PCT Filed: Nov. 24, 2017
(86) PCT No.: PCT/GB2017/053535

§ 371 Date: May 23, 2019

(30) Foreign Application Priority Data
Nov. 25, 2016 (GB) 1619997.8

(51) Int. Cl. A61B 3/16; A61B 5/00; G01L 9/00

(52) U.S. Cl. CPC A61B 3/16; A61B 5/6821; A61B 2562/12; A61B 2562/0247; G01L 9/00

(57) ABSTRACT

A method of determining the intraocular pressure of an eye of a subject, the method comprising the steps of: providing a measurement device comprising a contact lens having a pressure sensor, introducing the measurement device into the eye of the subject, obtaining data from the measurement device whilst the eye of the subject is open, and using the data to determine the intraocular pressure of the eye.

The present invention relates to a method and apparatus for determining the properties of an eye. The method and apparatus are particularly suitable for determining the intraocular pressure of the eye. Aspects of the invention relate to methods of manufacturing such apparatus and parts thereof.

### BACKGROUND

There is a recognized need to measure the intraocular pressure (IOP) of an eye of a subject for diagnosis of conditions such as glaucoma. In the most severe cases, glaucoma leads to irreversible blindness. High IOP may be indicative of the onset of glaucoma and early indication of high or increasing IOP is critical in ensuring that patients are provided with effective preventative care or treatment.

It is known that an individual's IOP varies over a given time period (e.g. 24 hours) and it is influenced by factors including the level of physical activity of the individual and their psychological status. Consequently, IOP is ideally measured multiple times over a relatively long period (such as 24 hours) to account for this variance.

International patent application WO-A-2012/052765 describes a device and method for measuring IOP. The described device includes a corneal contact lens having a pressure sensor mounted in a recess or cavity in the contact lens. The pressure sensor is in the form of a pair of capacitor plates. The contact lens includes a protruding portion that presses against the cornea of the eye when the contact lens is worn. In use, when the eyelids of the wearer are closed (e.g. as the eye blinks), the contact lens is pushed against the eye so that the protruding portion deforms and causes the distance between the pair of capacitor plates to vary. As such, the pressure sensor is able to measure the deformation of the protruding portion, and this measurement is used to provide an indication of the wearer's IOP.

A blink cycle (i.e. the time for an eyelid to close and reopen) is typically around 300 ms and the maximum pressure exerted by the eyelid on the contact lens (and underlying eye) occurs around 75 ms into the cycle, and lasts for around 16 ms. Accordingly, devices such as that described in WO-A-2012/052765 are required to obtain data during the brief period when the eyelid is exerting pressure on the contact lens. Given that eye blinks are spontaneous events, their occurrence is largely unpredictable. Consequently, such devices are required to obtain data at a high frequency (such as 10 ms intervals) over a long time period in order to obtain data coinciding with the maximum eyelid pressure. This is a processor intensive task which will require additional analysis to filter the data.

It is an object of certain embodiments of the present invention to overcome certain disadvantages of the prior art.

It is an object of certain embodiments of the present invention to provide an improved method of determining IOP.

It is an object of certain embodiments of the present invention to provide an improved measurement device for determining IOP.

### BRIEF SUMMARY OF THE DISCLOSURE

In accordance with an aspect of the present invention there is provided a method of determining the intraocular pressure of an eye of a subject, the method comprising the steps of:
(i) providing a measurement device comprising a contact lens having a pressure sensor;
(ii) introducing the measurement device into the eye of the subject;
(iii) obtaining data from the measurement device whilst the eye of the subject is open; and
(iv) using the data obtained in step (iii) to determine the intraocular pressure of the eye.

The method may further comprise inducing an increase in the intraocular pressure in the eye, and wherein step (iii) includes periodically obtaining data over a time period following the induced increase in intraocular pressure. In certain embodiments, the time period may be at least 10 minutes, at least 20 minutes, or at least 30 minutes.

The method may comprise the step of using the data obtained in step (iii) to determine a level of drainage of aqueous through the trabecular meshwork of the eye.

The contact lens may have a profile configured so that surface tension of a tear film of the eye causes the contact lens to substantially conform to the topography of a portion of the eye, wherein the portion of the eye may include the cornea and anterior sclera of the eye.

In certain embodiments, step (iii) may comprise obtaining data at a frequency of once or less per second.

The contact lens may include a protrusion configured to deform against the surface of the eye as the contact lens is urged against the eye, where deformation of the protrusion is measurable by the pressure sensor.

The contact lens may be urged against the eye by surface tension of a tear film of the eye and/or a negative pressure between the contact lens and the eye.

In certain embodiments, the pressure sensor comprises a reactive circuit element. In particular, the reactive circuit element may comprise one of a capacitor, an inductor, or a resistive element. In embodiments where the reactive circuit element is an inductor, the inductor may comprise an induction coil and/or the resistive element comprises a piezoresistance sensor.

In accordance with another aspect of the present invention, there is provided a method of manufacturing a pressure sensor for a contact lens measurement device, the method comprising:
providing a capacitor plate having a plurality of holes therethrough;
urging a diaphragm material through at least some of the plurality of holes; and
subsequently curing the diaphragm material to form a flexible diaphragm that is fixed to the capacitor plate.

Providing a capacitor plate having a plurality of holes therethrough may comprise laser cutting a plurality of holes in the capacitor plate.

Urging the diaphragm material through at least some of the plurality of holes may comprise compressing the capacitor plate against the diaphragm material.

In certain embodiments, the diaphragm material may comprise an addition-cured polymer.

In accordance with another aspect of the present invention, there is provided a method of manufacturing a contact lens measurement device comprising:
manufacturing a pressure sensor as described above;
providing a contact lens; and
assembling the pressure sensor on or in the contact lens.

In accordance with another aspect of the present invention, there is provided a measurement device comprising:

a contact lens; and

a pressure sensor arranged on or in the contact lens, the pressure sensor including a capacitor plate and a flexible diaphragm, wherein the capacitor plate has a plurality of holes therethrough and a portion of the diaphragm extends through at least some of the plurality of holes to fix the diaphragm to the capacitor plate.

In certain embodiments, the diaphragm material may comprise an addition-cured polymer.

In accordance with another aspect of the present invention, there is provided a measurement device comprising:
a contact lens; and
a pressure sensor arranged on or in the contact lens, the pressure sensor having an inductive component and a flexible diaphragm that is movable relative to the inductive component, wherein the flexible diaphragm includes one or more magnetically permeable components embedded therein.

In certain embodiments, the inductive component may comprise an induction coil.

The one or more magnetically permeable components may comprise one or more magnetically permeable rings, beads or particles. The magnetically permeable components may comprise ferrite.

In accordance with another aspect of the present invention, there is provided a measurement device comprising:

a contact lens having a recess; and

a pressure sensor receivable in the recess;

wherein the pressure sensor forms an interference fit when received in the recess.

The recess may comprise a base and side walls, wherein the side walls may be inclined relative to the base by an angle less than 90°.

The contact lens may be made of a resilient material.

The pressure sensor may be shaped so as to correspond to a shape of the recess.

The pressure sensor comprises a reactive circuit element, wherein the reactive circuit element may comprise one of a capacitor, an inductor, or a resistive element. In embodiments where the reactive circuit element is an inductor, the inductor may comprise an induction coil and/or the resistive element comprises a piezoresistance sensor.

### BRIEF DESCRIPTION OF THE DRAWINGS

Embodiments of the invention are further described hereinafter with reference to the accompanying drawings, in which:

FIG. 1 shows a representation of a method according to an embodiment of the present invention;

FIGS. 2A and 2B show a view with and without pressure sensor, respectively, of a measurement device according to an embodiment of the present invention;

FIG. 3 shows a detailed view of a part of the measurement device of FIG. 2A;

FIG. 4 shows example plots of IOP for an eye with good outflow and an eye with poor outflow upon an increase in lop;

FIG. 5 shows a sub-assembly of a pressure sensor according to an embodiment of the present invention;

FIG. 6 shows a detailed view of a part of a measurement device according to an alternative embodiment of the present invention;

FIG. 7 shows a part of a measurement device according to a further alternative embodiment of the present invention; and

FIG. 8 shows an exploded view of a part of a measurement device according to a further alternative embodiment of the present invention.

### DETAILED DESCRIPTION

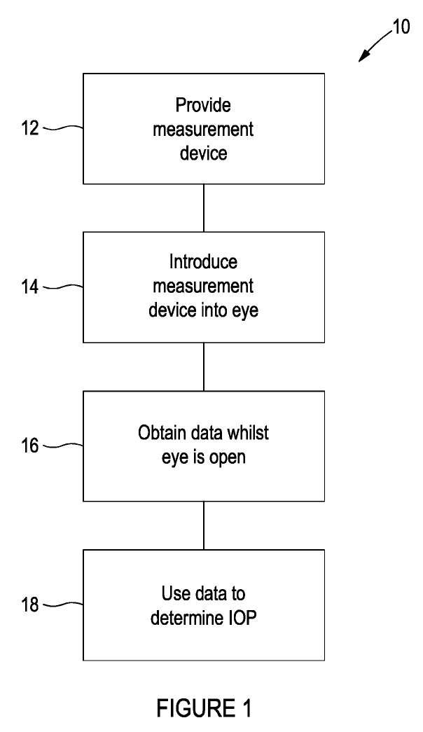

FIG. 1 shows a method 10 of determining the intraocular pressure (IOP) of an eye of a subject according to an embodiment of the present invention. The method comprises providing 12 a measurement device for measuring IOP, introducing 14 the measurement device into the eye of the subject, obtaining 16 data from the measurement device whilst the eye of the subject is open, and using 18 the obtained data to determine the IOP of the eye. The measurement device comprises a contact lens that may be worn by the subject and a pressure sensor (e.g. arranged in or on the contact lens).

It is found that, in contrast to prior art methods, external pressure such as eyelid pressure is not required to measure lop using a pressure sensor provided in or on a contact lens. Embodiments of the present invention rely on forces generated between the lens and the eye to pull the lens towards the eye. These forces are mainly caused by the surface tension resulting from the tear film. As such, measurements may be obtained whilst the eye is open, unlike prior art methods. For example, the effect of tear film surface tension at the lens periphery and/or negative pressure generated between the contact lens and the eye may cause the contact lens to conform to a portion of the eye (e.g. the cornea and/or anterior sclera). So-called “soft” contact lenses may be particularly suitable for demonstrating this effect. For the purpose of the present specification, the eye may be considered to be open if the eyelids of the eye are apart from one another.

The pressure sensor may be any suitable sensor that is capable of measuring changes in pressure. In certain embodiments the pressure sensor comprises a reactive circuit element such as a capacitor, an inductor or a resistive element. In certain embodiments of the invention, the pressure sensor is capable of measuring a movement or deflection and producing a signal indicative of the movement or deflection. In this manner, the deformation, movement or deflection may be measured to infer increases or decreases in pressure within the eye (i.e. IOP).

In embodiments in which the pressure sensor comprises a capacitor, the capacitor may include a pair of capacitor plates (or other elements) that are moveable relative to one another. For example, one plate may be provided on or in a flexible (or otherwise moveable) part, such as a diaphragm, such that it may move relative to the other plate, which may be held on or in a relatively rigid part. If the sensor is placed in proximity to the surface of the eye, deformation of the eye resulting from increases or decreases in IOP may cause the flexible part to move relative to the rigid part and therefore cause the distance between the capacitor plates to vary, thereby producing a signal indicative of the movement and the change in IOP. It will be apparent to the skilled reader that similar principles may be employed for other reactive elements such as inductors and resistive elements.

The pressure sensor may be “preloaded” when in place in the eye so that a baseline deflection or pressure may be established and changes beyond such a baseline are measured. For example, the measurement device may include a protrusion on a posterior surface that is configured to press against the corneal surface when the contact lens is worn (e.g. similar to the device described in WO-A-2012/052765). Contact between the protrusion and the corneal surface may provide the baseline (e.g. by causing the distance between the capacitor plates to reduce to a baseline distance). Increases or decreases in IOP may be measured by the pressure sensor relative to the baseline (e.g. as the distance between the capacitor plates increases or decreases relative to the baseline distance).

It is found that the actual extent of deflection (e.g. resulting in a change in distance between capacitor plates) is dependent on the fit between the contact lens and the eye, and the level of IOP.

It is observed that as IOP changes, the contact lens attempts to follow the change in topography of the eye. Whilst embodiments of the present invention are not dependent on external pressure provided by the eyelids, such pressure may be useful in stabilising the contact lens on the eye between measurements. For example, after one eyelid blink, the distance between capacitor plates may decrease by a small amount as the contact lens is pushed against the eye. If IOP remains constant thereafter, the distance will remain the same after each subsequent eyelid blink.

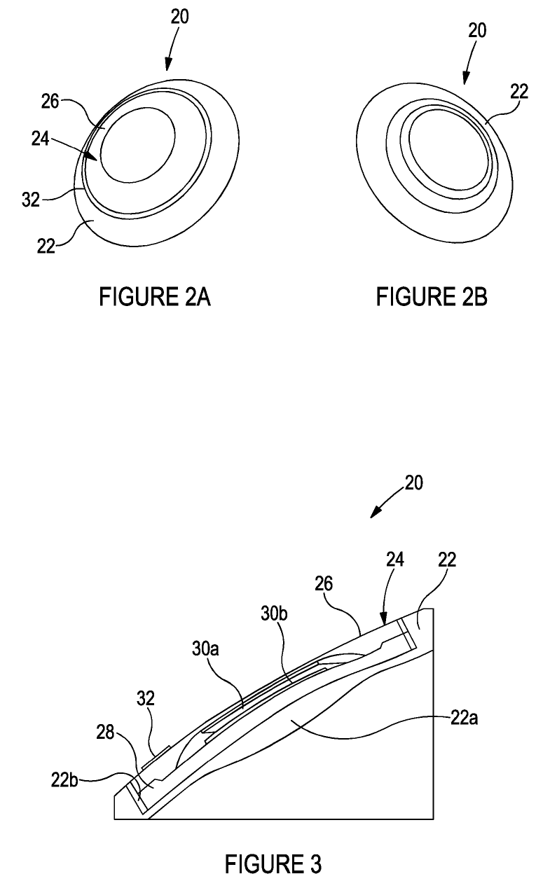

FIGS. 2A and 2B show a view with and without a pressure sensor, respectively, of a measurement device 20 according to an embodiment of the present invention. The measurement device 20 includes a contact lens 22 and a pressure sensor 24. FIG. 3 shows a cross sectional view through the measurement device 20 in which it can be seen that the pressure sensor 24 is received in a recess 22b of the contact lens 22 and includes a rigid part 26 and a flexible part 28 that is moveable relative to the rigid part 26. A first capacitor plate 30a is mounted to the rigid part 26 and a second capacitor plate 30b is mounted to the flexible part 28 such that the distance between the capacitor plates 30a,30b is variable in response to movement of the flexible part 28 relative to the rigid part 26. In alternative embodiments, a combed arrangement of capacitive elements may be utilized in place of a pair of capacitor plates.

The measurement device 20 includes a sensor coil 32 that is communicably coupled to the pressure sensor 24 and is arranged to transmit data obtained by the pressure sensor 24 to an external instrument (e.g. via a corresponding coil on or within the external instrument).

In the non-limiting embodiment shown in FIGS. 2A, 2B and 3, the portion of the contact lens 22 underlying the flexible part 28 of the pressure sensor 24 includes a protrusion 22a that protrudes beyond the profile of the adjacent parts of the contact lens 22. When the measurement device 20 is introduced into the eye of the subject, the protrusion 22a presses against the corneal surface of the eye so as to reduce the distance between the capacitor plates 30a,30b. As such, the presence of the protrusion may serve to provide the baseline described above.

Whilst the pressure sensor 24 forms a circle extending around a central portion of the contact lens 22 in the embodiment shown in FIGS. 2A and 3, in alternative embodiments, the pressure sensor may be provided as one or more discrete sensors arranged relative to the contact lens 22.

Methods according to embodiments of the present invention are particularly suitable for monitoring the reduction of IOP over a period of time. Such measurements may be useful in determining the extent of drainage or outflow of aqueous humour from within the eye. In glaucomatous eyes, the ability to drain aqueous humour through the trabecular meshwork of the eye can reduce or be lost entirely and thereby lead to an increase in IOP.

In accordance with certain embodiments, an increase in IOP may be deliberately induced. Examples of inducing an increase in IOP include pressure applied via a finger or ophthalmodynamometer. Data may be obtained from the measurement device periodically over a period of time following the induced increase in IOP. An eye with normal outflow will exhibit a drop in IOP following the induced increase over a period of around 17.9±11.17 min mmHg/μl at 40 mmHg and 4.51±2.69 min mmHg/μl at 20 mmHg. On the other hand, an eye with poor outflow will exhibit a more gradual decrease in IOP following the induced increase. Therefore periodic measurements over a time period of at least 10 minutes, at least 20 minutes or at least 30 minutes will likely be sufficient in permitting reliable measurement of outflow. In certain embodiments, periodic measurements may be obtained over a time period between 10 minutes and 1 hour. In preferable embodiments, the induced increase in IOP is also measured so as to provide a maximum comparative level from which IOP decreases.

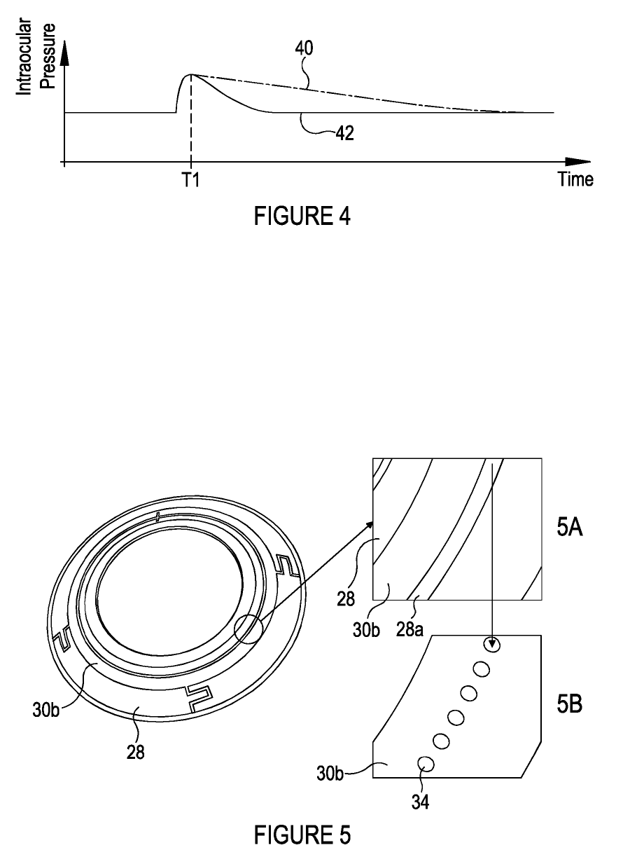

FIG. 4 shows an example plot of determined IOP following an induced IOP increase for an eye with good outflow and for an eye with poor outflow. In particular, the maximum IOP occurs at time T1 due to an induced IOP increase. Line 40 shows a gradual decrease in IOP following the initial increase and is indicative of the behavior of an eye with poor outflow. In contrast, line 42 shows a more rapid reduction in IOP following the initial increase and is representative of the behavior of an eye with good outflow.

FIG. 5 shows a non-limiting embodiment of a sub-assembly of the pressure sensor 24. The sub-assembly includes a flexible diaphragm 28 and a capacitor plate 30b. Capacitor plate 30b may be considered to be equivalent to the second capacitor plate 30b described above in relation to FIG. 3 and the diaphragm 28 may be considered to be equivalent to the flexible part 28 described above in relation to FIG. 3 and the same reference numerals are used for consistency. The capacitor plate 30b is fixed to the diaphragm 28. Insert FIG. 5A shows a detailed view of a part of the sub-assembly in which the diaphragm 28 is seen to extend along a track 28a over part of the capacitor plate 30b. Insert FIG. 5B shows a portion of the capacitor plate 30b without any overlying diaphragm material (e.g. as it would appear prior to fabrication of the pressure sensor). As shown in Insert FIG. 5B, the capacitor plate 30b includes a plurality of holes. In certain embodiments, the plurality of holes 34 may be distributed along a central line around the entire capacitor plate 30b. In certain embodiments, the holes 34 may be produced by laser cutting the capacitor plate 30b. To form the sub-assembly, a diaphragm material may be urged through the holes 34 and subsequently cured to fix the capacitor plate 30b to the formed diaphragm 28. In certain embodiments, the capacitor plate 30b may be compressed against addition-cured polymer material or other similarly moldable elastic material (forming the diaphragm material) e.g. in an assembly jig. In such embodiments, the compression (e.g. within the jig) may force the addition cured polymer to take the desired form of the diaphragm 28, and for the addition-cured polymer material to be forced through the holes 38. When cured, the diaphragm 28 is effectively riveted to the capacitor plate 30b. The discrete spacing of the holes 34 may advantageously reduce the interaction between the capacitor plate 30b and the diaphragm 28 whilst facilitating a secure connection.

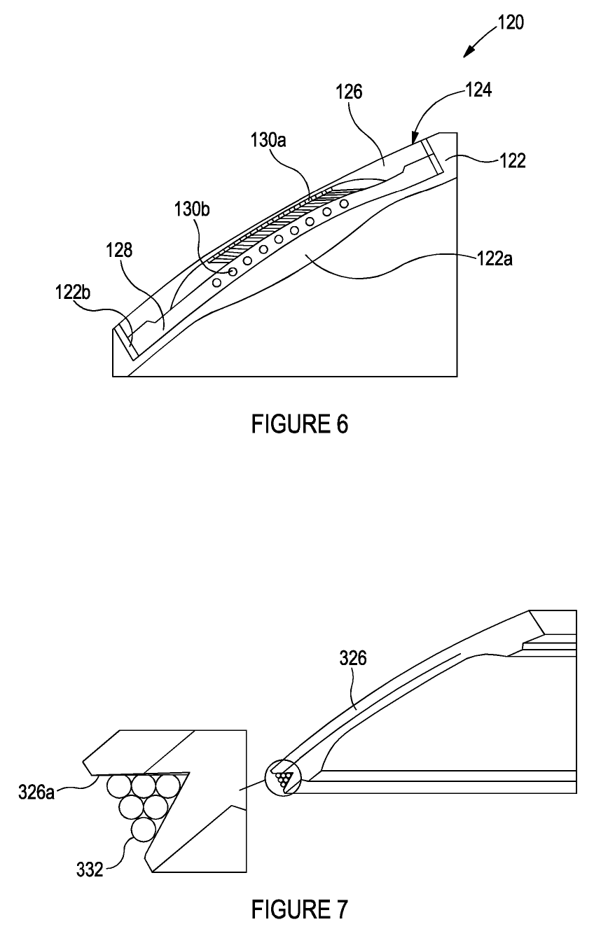

FIG. 6 shows a measurement device 120 according to an alternative embodiment of the present invention. The measurement device 120 shares many features with the measurement device 20 described above with reference to FIG. 3. Equivalent or similar features are identified using common numerals preceded by a ‘1’. The measurement device 120 of FIG. 6 differs to the measurement device 20 of FIG. 3 in that the capacitor plates 30a,30b are replaced with an induction coil 130a and a plurality of ferrite rings 130b. In the embodiment shown in FIG. 6, the induction coil 130a is provided (e.g. deposited) on an internal surface of the rigid part 126, while the ferrite rings 130b are embedded within the flexible part 128. The resulting pressure sensor 124 measures inductance rather than capacitance, in contrast to the measurement device 20 described above. Despite this difference, the operation of the pressure sensor 124 is similar. In particular, movements of the flexible part 128 relative to the rigid part 126 due to changes in IOP cause the distance between the inductance coil 130a and ferrite rings 130b to change and result in a change in inductance/impedance. This change in inductance/impedance may be used to determine the IOP of the eye.

In alternative embodiments, the induction coil 130a may be replaced with any suitable inductor. Additionally or alternatively, the ferrite rings 130b may be replaced with other ferrite components such as beads or particles that may be provided on or in the flexible part 128. In certain embodiments, the inductor may be provided on or in the flexible part 128 and the ferrite component may be provided on or in the rigid part.

In certain embodiments, the ferrite component may be replaced with a piezoresistive material and the resulting pressure sensor may measure piezoresistance in order to permit IOP to be determined.

FIG. 7 shows a sub-assembly of a pressure sensor according to an alternative embodiment of the present invention. The sub-assembly includes a rigid part 326 with a groove 326a along an edge. Within the groove 326a is wound a sensor coil 332 for transmitting data measured by the pressure sensor to an external device. The pressure sensor may be received in a recess of the contact lens so that the sensor coil 332 is housed within the groove 326a between the rigid part 326 and the contact lens. In alternative embodiments, the groove may be provided on other parts of the pressure sensor so as to house the sensor coil between the pressure sensor and the contact lens when the pressure sensor is received in the recess of the contact lens. Housing the sensor coil in the groove may advantageously simplify the process of manufacturing the measurement device.

In alternative embodiments, the sensor coil may be provided on one of the capacitor plates, if present. For example, the coil and capacitor plate could be built up by photolithography. Alternatively, laser etching or laser direct structuring (LDS) may be used to form the coil and a capacitor plate on one of the pressure sensor components, such as the rigid part.

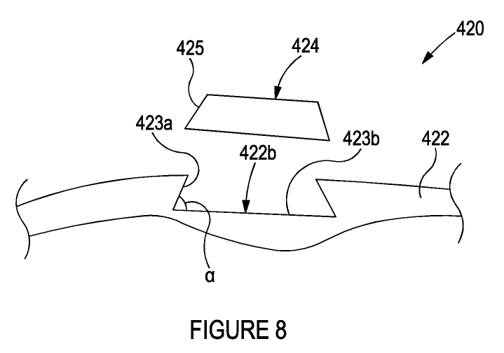

FIG. 8 shows a part of a measurement device 420 according to an alternative embodiment of the present invention. The measurement device 420 includes a contact lens 422 and a pressure sensor 424. The contact lens 422 and/or pressure sensor 424 may be in accordance with any of the above described embodiments. The contact lens 422 has a recess 422b for receiving the pressure sensor 424. The recess 422b is defined by side walls 423a and a base 423b. The side walls 423a are inclined at an angle α relative to the base 423b, where the angle α is less than 90°. As such, the recess 422b narrows in a direction away from the base 423b. Given that the contact lens 422 is comprised of a flexible material, the narrow part of the recess 422b may flex to permit the pressure sensor 424 to be received and flex back to retain the pressure sensor 424 therein. The pressure sensor 424 may additionally have inclined walls 425 that may correspond to the side walls 423a of the recess 422b so as to create a close fit when the pressure sensor 424 is received in the recess 422b. Advantageously, this simplifies the process of fixing of the pressure sensor 424 into the contact lens 422. In alternative embodiments, any other formation that permits an interference fit between the pressure sensor 424 and recess 422b may be employed, particularly where the resilience of the contact lens 422 is utilized to retain the pressure sensor 424 in the recess 422b.

In any embodiment, the pressure sensor may be provided with a moisture barrier to reduce or prevent moisture ingress. Moisture ingress could lead to reduced performance of the pressure sensor, e.g. through short circuiting certain components. In certain embodiments, the moisture barrier may be provided by petroleum-based jelly or a coating such as silicon oxide, aluminium oxide or another material that is impenetrable to water. The moisture barrier may be applied to the external surface of the pressure sensor to provide temporary protection against moisture ingress. In other embodiments, a more permanent solution may be implemented by depositing the moisture barrier on the rigid part 26 to cover the capacitor plate 30a and sensor coil 32, thereby isolating these components and preventing a short-circuit between capacitor plates 30a and 30b. The inclusion of a moisture barrier would additionally permit the pressure sensor to be stored in a wet environment, if desired.

Any features of the above-described embodiments may be combined in any suitable combination so as to provide further embodiments (e.g. of measurement devices) of the present invention.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of determining the intraocular pressure of an eye of a subject, the method comprising the steps of:
(i) providing a measurement device comprising a contact lens having a pressure sensor;
(ii) introducing the measurement device into the eye of the subject;
(iii) obtaining data from the measurement device whilst the eye of the subject is open; and
(iv) using the data obtained in step (iii) to determine the intraocular pressure of the eye.

2. The method of any preceding claim, further comprising inducing an increase in the intraocular pressure in the eye, and wherein step (iii) includes periodically obtaining data over a time period following the induced increase in intraocular pressure.

3. The method of claim 2, wherein the time period is at least 10 minutes, at least 20 minutes, or at least 30 minutes.

4. The method of claim 2 or 3, wherein the method comprises using the data obtained in step (iii) to determine a level of drainage of aqueous through the trabecular meshwork of the eye.

5. The method of any preceding claim, where the contact lens has a profile configured so that surface tension of a tear film of the eye causes the contact lens to substantially conform to the topography of a portion of the eye.

6. The method of claim 5, wherein the portion of the eye includes the cornea and anterior sclera of the eye.

7. The method of any preceding claim, wherein step (iii) comprises obtaining data at a frequency of once or less per second.

8. The method of any preceding claim, wherein the contact lens includes a protrusion configured to deform against the surface of the eye as the contact lens is urged against the eye, where deformation of the protrusion is measurable by the pressure sensor.

9. The method of claim 8, where the contact lens is urged against the eye by surface tension of a tear film of the eye and/or a negative pressure between the contact lens and the eye.

10. The method of any preceding claim, wherein the pressure sensor comprises a reactive circuit element.

11. The method of claim 10, wherein the reactive circuit element comprises one of a capacitor, an inductor, or a resistive element.

12. The method of claim 11, wherein the inductor comprises an induction coil and/or the resistive element comprises a piezoresistance sensor.

13. A method of manufacturing a pressure sensor for a contact lens measurement device, the method comprising:
providing a capacitor plate having a plurality of holes therethrough;
urging a diaphragm material through at least some of the plurality of holes; and
subsequently curing the diaphragm material to form a flexible diaphragm that is fixed to the capacitor plate.

14. The method of claim 13, wherein providing a capacitor plate having a plurality of holes therethrough comprises laser cutting a plurality of holes in the capacitor plate.

15. The method of claim 13 or 14, wherein urging the diaphragm material through at least some of the plurality of holes comprises compressing the capacitor plate against the diaphragm material.

16. The method of any of claims 13 to 15, wherein the diaphragm material comprises an addition-cured polymer.

17. A method of manufacturing a contact lens measurement device comprising:
manufacturing a pressure sensor in accordance with any of claims 13 to 16;
providing a contact lens; and
assembling the pressure sensor on or in the contact lens.

18. A measurement device comprising:
a contact lens; and
a pressure sensor arranged on or in the contact lens, the pressure sensor including a capacitor plate and a flexible diaphragm, wherein the capacitor plate has a plurality of holes therethrough and a portion of the diaphragm extends through at least some of the plurality of holes to fix the diaphragm to the capacitor plate.

19. The measurement device of claim 18, wherein the diaphragm material comprises an addition-cured polymer.

20. A measurement device comprising:
a contact lens; and
a pressure sensor arranged on or in the contact lens, the pressure sensor having an inductive component and a flexible diaphragm that is movable relative to the inductive component, wherein the flexible diaphragm includes one or more magnetically permeable components embedded therein.

21. The measurement device of claim 20, wherein the inductive component comprises an induction coil.

22. The measurement device of claim 20 or 21, wherein the one or more magnetically permeable components comprise one or more magnetically permeable rings, beads or particles.

23. The measurement device of any of claims 20 to 22, wherein the magnetically permeable components comprises ferrite.

24. A measurement device comprising:
a contact lens having a recess; and
a pressure sensor receivable in the recess;
wherein the pressure sensor forms an interference fit when received in the recess.

25. The measurement device of claim 24, wherein the recess comprises a base and side walls, wherein the side walls are inclined relative to the base by an angle less than 90°.

26. The measurement device of claim 24 or 25, wherein the contact lens is made of a resilient material.

27. The measurement device of any of claims 24 to 26, wherein the pressure sensor is shaped so as to correspond to a shape of the recess.

28. The measurement device of any of claims 24 to 27, wherein the pressure sensor comprises a reactive circuit element.

29. The measurement device of claim 28, wherein the reactive circuit element comprises one of a capacitor, an inductor, or a resistive element.

30. The measurement device of claim 29, wherein the inductor comprises an induction coil and/or the resistive element comprises a piezoresistance sensor.

31. A method of determining the intraocular pressure of an eye of a subject substantially as hereinbefore described with reference to the accompanying drawings.

32. A method of manufacturing contact lens measurement device or a pressure sensor for a contact lens measurement device substantially as hereinbefore described with reference to the accompanying drawings.

33. A measurement device substantially as hereinbefore described with reference to the accompanying drawings.
